(19) United States
(12) Patent Application Publication
(10) Pub. No.: US 2019/0194154 A1
(43) Pub. Date: Jun. 27, 2019

(54) Polymorphic Forms Of Vortioxetine Hydrobromide Tert-Butanolate

(71) Applicant: AMNEAL PHARMACEUTICALS COMPANY GMBH, Steinhausen (CH)

(72) Inventors: Virendra Kumar Agarwal, Ahmedabad (IN); Joseph Prabahar Koilpillai, Tirunelveli (IN); Abhay Subodhbhai Maheta, Ahmedabad, Gujarat (IN); Rajesh Gangarambhai Rupala, Dist-Morbi (IN); Pankaj Chaganbhai Butani, Rajkot, Gujarat (IN); Parag Vrujlal Ajudia, Dist-Jamnagar, Gujarat (IN); Chirag Mansukhbhai Jethva, Nadiad, Gujarat (IN)

(21) Appl. No.: 16/071,263

(22) PCT Filed: Jan. 19, 2017
(86) PCT No.: PCT/EP2017/051103

§ 371 Date: Jul. 19, 2018

(30) Foreign Application Priority Data
Jan. 20, 2016 (IN) 201621002145

(51) Int. Cl. C07D 295/096

(52) U.S. Cl. CPC C07D 295/096; C07B 2200/13

(57) ABSTRACT

The present invention provides novel polymorphic forms of vortioxetine hydrobromide (I).

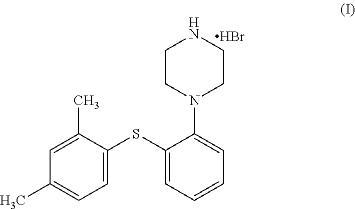 [SMILES: Cc1ccc(Sc2ccccc2N2CCNCC2)c(C)c1 ; Br]

### FIELD OF INVENTION

The present invention relates to novel polymorphic forms of vortioxetine hydrobromide (I). The present invention also relates to the process for the preparation of novel polymorphic forms of vortioxetine hydrobromide.

### BACKGROUND OF INVENTION

Vortioxetine hydrobromide is used as antidepressant. It is indicated for the treatment of major depressive disorder (MDD). Vortioxetine hydrobromide is known by chemical name 1-[2-(2,4-Dimethylphenylsulfanyl)phenyl]-piperazine hydrobromide. Vortioxetine hydrobromide is marketed in USA by Takeda Pharms under trade name Brintellix® in the form of oral tablet of 5 mg, 10 mg, 15 mg and 20 mg eq. base. Vortioxetine hydrobromide is represented by following structure.

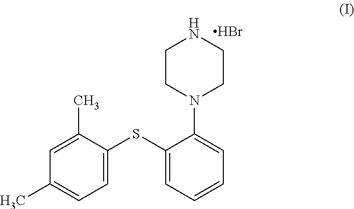 [SMILES: Br ; Cc1ccc(Sc2ccccc2N2CCNCC2)c(C)c1]

Vortioxetine is first time disclosed in U.S. Pat. No. 7,144,884 B2. This patent discloses process for preparation of vortioxetine.

U.S. Pat. No. 8,722,684 B2 disclose crystalline forms of vortioxetine free base and crystalline form α (alpha), β (beta), γ (gamma), hemihydrates of vortioxetine hydrobromide. It also discloses crystalline vortioxetine ethylacetate solvate.

U.S. Pat. No. 8,598,348 B2 discloses isopropanol solvate of vortioxetine hydrobromide.

WO 2014/044721 A1 discloses delta form and hydrate form of vortioxetine hydrobromide.

WO 2014/177491 A1 discloses amorphous vortioxetine hydrobromide in association with an adsorbent.

WO 2015/044963 A1 discloses amorphous vortioxetine hydrobromide and its solid dispersion.

WO 2015/114395 A1 discloses polymorphic forms of vortioxetine salts formed with salicylic acid, citric acid, malonic acid, oxalic acid, L-malic acid, benzenesulfonic acid, acetic acid, succinic acid, L-amygdalic acid.

Polymorphs are solid forms which share the molecular formula of the compound from which the crystals are made up, however they may different physical properties such as e.g. chemical and physical stability, hygroscopicity, solubility, dissolution rate, morphology or bioavailability. It is always required that the polymorphic forms has to be stable and suitable for formulation purpose. The present invention provides new polymorphic forms of vortioxetine hydrobromide solvate. The present invention provides tertiary butanol and 2-butanol solvate forms of vortioxetine hydrobromide.

### SUMMARY OF THE INVENTION

The present invention relates to solvates of tertiary butanol and 2-butanol of vortioxetine hydrobromide

In one aspect, the present invention relates to tertiary butanol (tert-butanol) solvate of vortioxetine hydrobromide form R1 and form R2.

Vortioxetine hydrobromide tert-butanol solvate form R1 can be characterized by x-ray powder diffraction (XRPD) patterns having peaks at about 6.7, 8.6, 15.9 and 19.1±0.2°2θ; differential scanning calorimetry (DSC) thermogram having endothermic peaks at about 129.22° C. and at about 226.9° C.; thermogravimetric analysis (TGA) with weight loss of about 16.48%.

Vortioxetine hydrobromide tert-butanol solvate form R2 can be characterized by XRPD patterns having peaks at about 7.5, 10.0, 18.1 and 20.2±0.2°2θ; DSC thermogram having endothermic peaks at about 132.80° C. and at about 226.51° C.; TGA with weight loss of about 16.45%.

In another aspect, the present invention relates to 2-butanol solvate of vortioxetine hydrobromide form R3 and form R4.

Vortioxetine hydrobromide 2-butanol solvate form R3 can be characterized by XRPD patterns having peaks at about 6.6, 8.6, 15.9 and 19.1±0.2°2θ; DSC thermogram having endothermic peaks at about 96.34° C., 227.75° C. and at about 234.21° C.; TGA with weight loss of about 15.72%.

Vortioxetine hydrobromide 2-butanol solvate form R4 can be characterized by XRPD patterns having peaks at about 7.8, 10.1, 18.5 and 20.4±0.2°2θ; DSC thermogram having endothermic peaks at about 92.36° C., 226.18° C. and at about 230.77° C.; TGA with weight loss of about 16.30%.

In further embodiment, the invention provide processes for the preparation of vortioxetine hydrobromide solvate form R1, R2, R3 and R4.

### BRIEF DESCRIPTION OF THE DRAWINGS

FIG. 1: X-ray powder diffractogram (XRPD) of vortioxetine hydrobromide tert-butanol solvate form R1

FIG. 2: Differential Scanning calorimetry (DSC) thermogram of vortioxetine hydrobromide tert-butanol solvate form R1

FIG. 3: Thermogravimetric analysis (TGA) curve of vortioxetine hydrobromide tert-butanol solvate form R1

FIG. 4: X-ray powder diffractogram (XRPD) of vortioxetine hydrobromide tert-butanol solvate form R2

FIG. 5: Differential Scanning calorimetry (DSC) thermogram of vortioxetine hydrobromide tert-butanol solvate form R2

FIG. 6: Thermogravimetric analysis (TGA) curve of vortioxetine hydrobromide tert-butanol solvate form R2

FIG. 7: X-ray powder diffractogram (XRPD) of vortioxetine hydrobromide 2-butanol solvate form R3

FIG. 8: Differential Scanning calorimetry (DSC) thermogram of vortioxetine hydrobromide 2-butanol solvate form R3

FIG. 9: Thermogravimetric analysis (TGA) curve of vortioxetine hydrobromide 2-butanol solvate form R3

FIG. 10: X-ray powder diffractogram (XRPD) of vortioxetine hydrobromide 2-butanol solvate form R4

FIG. 11: Differential Scanning calorimetry thermogram (DSC) of vortioxetine hydrobromide 2-butanol solvate form R4

FIG. 12: Thermogravimetric analysis (TGA) curve of vortioxetine hydrobromide 2-butanol solvate form R4

### DETAIL DESCRIPTION OF INVENTION

A solvate may be defined as a compound formed by solvation, for example as a combination of solvent molecules with molecules or ions of a solute. Well known solvent molecules include water, alcohols and other polar organic solvents. Alcohols include methanol, ethanol, n-propanol, isopropanol, n-butanol, isobutanol, 2-butanol and tert butanol.

The novel polymorphic forms of vortioxetine hydrobromide of the present invention can be characterized by x-ray powder diffraction (XRPD) or differential scanning calorimetry (DSC). The novel crystalline forms of the present invention can exist as solvates, especially solvates with tert-butanol and 2-butanol.

Analytical Methods

Characterization by Powder X-Ray Diffraction

Analytical method: Powder X-ray Diffraction can be performed using PANALYTICAL ExpertPro DY666, the powder X-ray diffraction pattern was measured at room temperature using a Cu Kα filled tube (45 kV, 40 mA) as the X-ray source with a wide-angle goniometer, a ½° scattering slit, an programable divergence slit, and a x'celerator detector [1]. Data collection was done in 20 continuous scan mode at a scan speed of 0.047747/s in scan steps of 0.0083556° in the range of 3° to 45°. Cu radiation of λ=1.5405 A° was used.

Differential scanning calorimetry (DSC) and thermogravimetric analysis (TGA) thermograms presented herein were obtained by methods known in the art. Differential scanning calorimetric (DSC) analysis was performed with a Shimadzu DSC 60 calorimeter. Samples of about 2 to about 6 milligrams, held in aluminum crucible, were analyzed at a heating rate of 10° C. per minute in a loosely closed pan under nitrogen atmosphere.

Thermogravimetric analysis (TGA) was performed using a Shimadzu DTG60 thermobalance. TGA traces reflect transitions that involve either a loss or gain of mass. Samples of about 3 to about 6 milligrams were analyzed at a heating rate of 10° C. per minute in nitrogen atmosphere.

In one embodiment, the present invention provides tert-butanol solvate of vortioxetine hydrobromide form R1 and form R2.

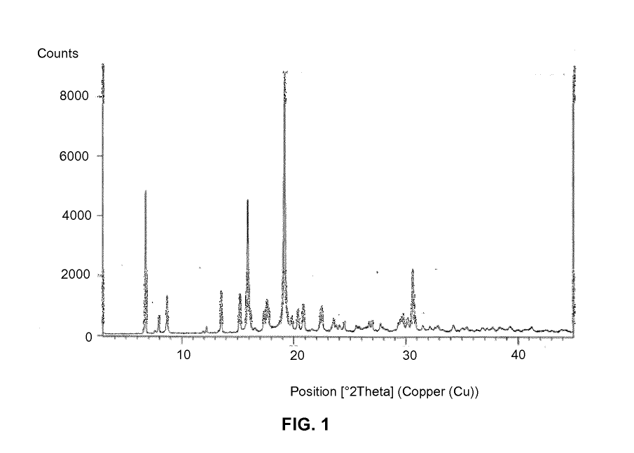

Vortioxetine hydrobromide form R1 can be characterized by XRPD patterns having peaks at about 6.7, 8.6, 15.9 and 19.1±0.2°2θ. vortioxetine hydrobromide Form R1 can be further characterized by XRPD reflections of peaks at about 6.7, 7.9, 8.6, 13.5, 15.2, 15.9, 17.6, 19.1, 20.3, 20.8, 22.4 and 30.6±0.2°2θ. A typical XRPD of vortioxetine hydrobromide form R1 as tert-butanol solvate is shown in FIG. 1.

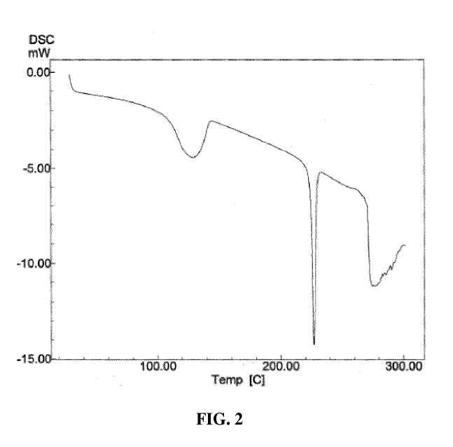

The DSC thermogram of vortioxetine hydrobromide form R1 is shown in FIG. 2. The DSC thermogram of vortioxetine hydrobromide form R1 is characterized by endothermic peaks at about 129.22° C. and at about 226.9° C.

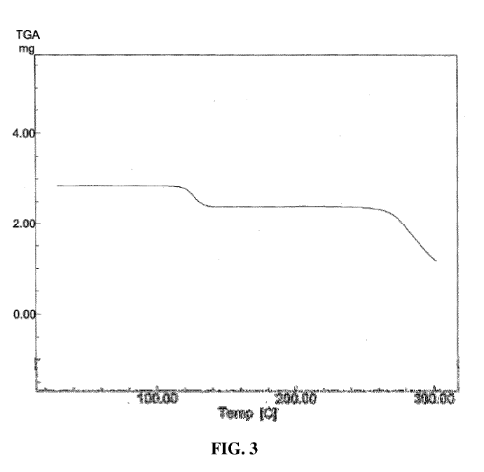

Vortioxetine hydrobromide form R1 shows weight loss of about 16.48% in TGA in the temperature range of between 95° C. and about 150° C. TGA for vortioxetine hydrobromide Form R1 as tert-butanol solvate is shown in FIG. 3

In another aspect, the present invention provides a process for preparation of vortioxetine hydrobromide solvate form R1 comprising the steps of:

(a) providing a solution of vortioxetine free base in tert-butanol or mixture of solvent containing tert-butanol

(b) adding hydrobromic acid to the above solution and

(c) isolating the resulting solid

Vortioxetine free base is dissolved in tert-butanol or in mixture of solvent containing tert-butanol. The mixture of solvent may be prepared by taking one or more cosolvent with tert-butanol. The cosolvent is selected from organic solvent such as chlorinated hydrocarbons such as dichloromethane, dichloroethane, chloroform or carbon tetrachloride; aromatic hydrocarbon such as toluene, xylene; methyl tertbutyl ether (MTBE); nitrile such as acetonitrile; ester such as ethylacetate, isopropyl acetate; ketone such as acetone, methyl isobutyl ketone (MIBK), methyl ethyl ketone (MEK); polar aprotic such as N,N-dimethylformamide (DMF), dimethyl sulfoxide (DMSO), dimethyl acetamide (DMAc), N-methylpyrrolidone (NMP); polar protic such as alcoholic solvent C₁₋₄ alcohol such as methanol, ethanol, isopropanol, propanol, butanol and the like. The dissolution may be done at ambient temperature or at elevated temperature by heating the mixture at 40° C. to reflux. Tert-butanol is taken in 5 to 20 volume by weight of the compound. The clear solution is filtered through hyflo bed to remove any fine particles. The filtrate is heated at 40° C. to reflux, preferably at 50-60° C. To this clear hot solution, hydrobromic acid is added. Hydrobromic acid may be provided as a gas or as a solution in an organic or aqueous solvent. For example aqueous hydrobromic acid, hydrobromic acid in acetic acid or hydrobromic acid gas in organic solvent can be used. It is taken in stoichiometric amount ranging from 1 to 2 mol equivalent of the compound. The reaction mixture is heated from 40° C. to reflux, preferably at 50-60° C. for 1 to 3 hour. The reaction mixture is gradually cooled to ambient temperature 25-35° C. and stirred for 1 to 3 hour. The precipitates are obtained. This process also encompass use of seed of form R1 to induce crystallization. The resulting precipitates were isolated from the suspension by the methods known in the art such as filtration or centrifugation. The solid is dried at 40-50° C. The solid was analysed by XRPD, DSC and TGA. The analytical data revealed that it is tert-butanolsolvate of vortioxetine hydrobromide. It is designated as form R1. The vortioxetine free base used in above process is having HPLC purity 99.0 or above.

In another aspect, the present invention provides a process for preparation of vortioxetine hydrobromide solvate form R1 comprising the steps of:

(a) providing a suspension of vortioxetine hydrobromide in tert-butanol or mixture of solvent containing tert-butanol

(b) isolating the resulting solid

In this process, vortioxetine hydrobromide is added to tert-butanol or mixture of solvent containing tert-butanol to provide suspension. The mixture of solvent may be prepared by taking one or more cosolvent with tert butanol. The cosolvent is as defined above. The suspension is heated at 40° C. to reflux, preferably at 50-60° C. for 1 to 3 hour. Tert-butanol is taken in 5 to 20 volume by weight of the compound. The reaction mixture does not become clear solution. The reaction mixture is gradually cooled to ambient temperature 25-35° C. and stirred for 1 to 3 hour. The solid was isolated from the suspension by the methods known in the art such as filtration or centrifugation. The solid is dried at 40-50° C. under vacuum. The solid was analysed by XRPD, DSC and TGA. The analytical data revealed that it is tert-butanolsolvate Form R1 of vortioxetine hydrobromide. The vortioxetine hydrobromide used in above process is having HPLC purity 99.0 or above.

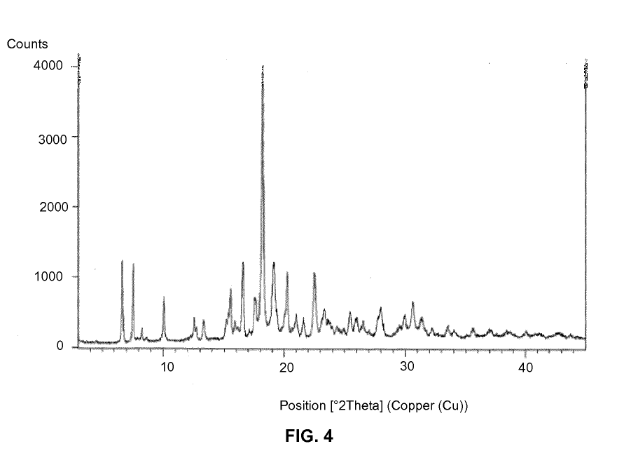

Vortioxetine hydrobromide form R2 can be characterized by XRPD patterns having peaks at about 7.5, 10.0, 18.1 and 20.2±0.2°2θ. Vortioxetine hydrobromide form R2 can be further characterized by XRPD reflections at 6.6, 7.5, 10.0, 13.3, 16.5, 18.1, 19.0, 19.1, 20.2, 22.5, 28.0 and 30.6±0.2°2θ. A typical XRPD of vortioxetine hydrobromide form R2 as tert-butanol solvate is shown in FIG. 4.

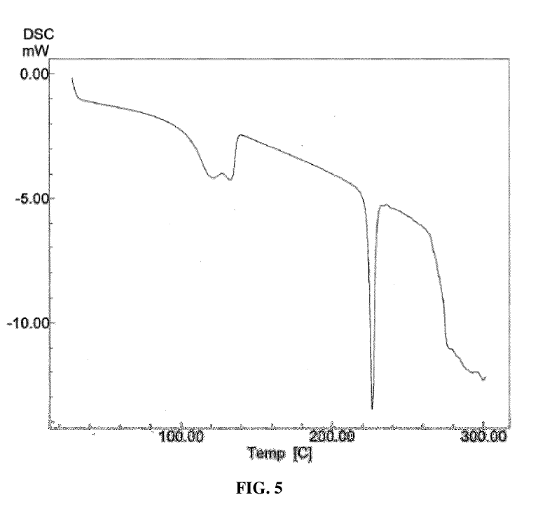

The DSC thermogram of vortioxetine hydrobromide form R2 is shown in FIG. 5. The DSC thermogram of vortioxetine hydrobromide form R2 is characterized by endothermic peaks at about 132.80° C. and at about 226.51° C.

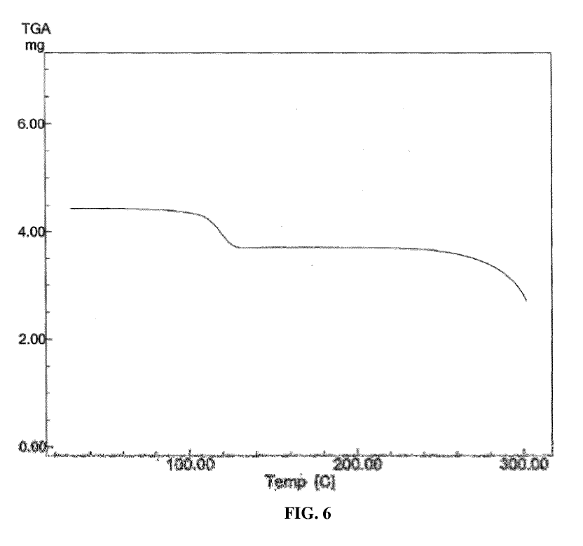

Vortioxetine hydrobromide form R2 shows weight loss of about 16.45% in TGA in the temperature range of between 95° C. and about 150° C. TGA for vortioxetine hydrobromide form R2 as tert-butanol solvate is shown in FIG. 6

The present invention relates to a process for preparation of vortioxetine hydrobromide solvate form R2 comprising the steps of:

(a) providing a suspension of vortioxetine free base in tert-butanol or mixture of solvent containing tert butanol

(b) adding hydrobromic acid to the above suspension and

(c) isolating the resulting solid

Vortioxetine free base is added in tert-butanolor mixture of solvent containing tert-butanol to provide suspension. The mixture of solvent may be prepared by taking one or more cosolvent with tert butanol. The cosolvent is selected from organic solvent such as chlorinated hydrocarbons such as dichloromethane, dichloroethane, chloroform or carbon tetrachloride; aromatic hydrocarbon such as toluene, xylene; methyl tertbutyl ether (MTBE); nitrile such as acetonitrile; ester such as ethylacetate, isopropyl acetate; ketone such as acetone, methyl isobutyl ketone (MIBK), methyl ethyl ketone (MEK); polar aprotic such as N,N-dimethylformamide (DMF), dimethyl sulfoxide (DMSO), dimethyl acetamide (DMAc), N-methylpyrrolidone (NMP); polar protic such as alcoholic solvent C₁₋₄ alcohol such as methanol, ethanol, isopropanol, propanol, butanol and the like. The suspension is heated at 50-60° C. to reflux for 20-30 min. The reaction mixture does not become clear solution. Tert-butanol is taken in 5 to 20 volume by weight of the compound. To this hot suspension hydrobromic acid is added. Hydrobromic acid may be provided as a gas or as a solution in an organic or aq. solvent. For example aq. hydrobromic acid, hydrobromic acid in acetic acid or hydrobromic acid gas in organic solvent can be used. It is taken in stoichiometric amount ranging from 1 to 2 mol equivalent of the compound. The reaction mixture is heated from 40° C. to reflux, preferably at 50-60° C. for 1 to 3 hour. The reaction mixture is gradually cooled to ambient temperature 25-35° C. and stirred for 1 to 3 hour. The resulting precipitates were isolated from the suspension by the methods known in the art such as filtration or centrifugation. The solid is dried at 40-50° C. under vacuum. The solid was analysed by XRPD, DSC and TGA. The analytical data revealed that it is tert-butanol solvate of vortioxetine hydrobromide. It is designated as from R2. The vortioxetine free base used in above process is having HPLC purity 99.5 or above.

In another aspect, the present invention provides 2-butanol solvate of vortioxetine hydrobromide form R3 and form R4.

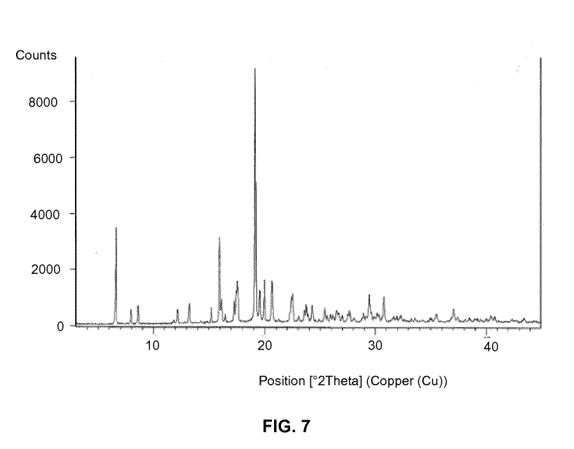

Vortioxetine hydrobromide form R3 can be characterized by XRPD patterns having peaks at about 6.6, 8.6, 15.9 and 19.1±0.2°2θ. Vortioxetine hydrobromide form R3 can be further characterized by XRPD reflections of peaks at 6.6, 7.9, 8.6, 13.2, 15.2, 15.9, 17.5, 19.1, 19.9, 20.6, 22.5 and 24.2±0.2°2θ. A typical XRPD of vortioxetine hydrobromide form R3 as 2-butanol solvate is shown in FIG. 7.

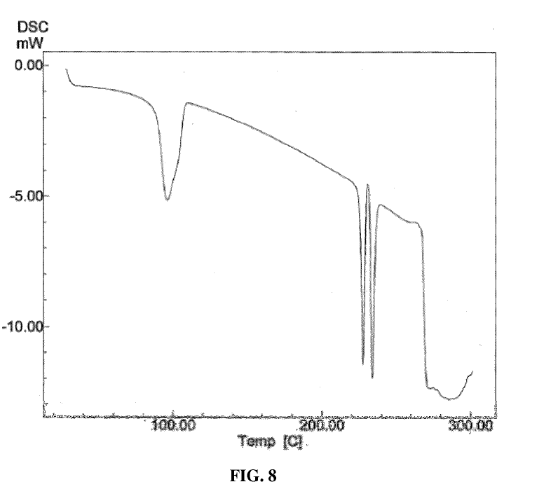

The DSC thermogram of vortioxetine hydrobromide form R3 is shown in FIG. 8. The DSC thermogram of vortioxetine hydrobromide form R3 is characterized by endothermic peaks at about 96.34° C., 227.75° C. and at about 234.21° C.

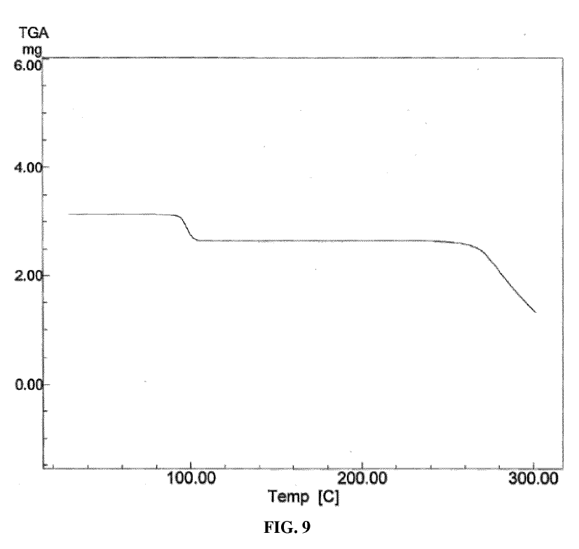

Vortioxetine hydrobromide form R3 shows weight loss of about 15.72% in TGA in the temperature range of between 80° C. and about 120° C. TGA for vortioxetine hydrobromide form R3 as 2-butanol solvate is shown in FIG. 9.

In another aspect, the present invention relates to a process for preparation of vortioxetine hydrobromide solvate form R3 comprising the steps of:

(a) providing a solution of vortioxetine free base in 2-butanol or mixture of solvent containing 2-butanol

(b) adding hydrobromic acid to the above solution and

(c) isolating the resulting solid

Vortioxetine free base is dissolved in 2-butanol or in mixture of solvent containing 2-butanol. The mixture of solvent may be prepared by taking one or more cosolvent with 2-butanol. The cosolvent is selected from organic solvent such as chlorinated hydrocarbons such as dichloromethane, dichloroethane, chloroform or carbon tetrachloride; aromatic hydrocarbon such as toluene, xylene; methyl tertbutyl ether (MTBE); nitrile such as acetonitrile; ester such as ethylacetate, isopropyl acetate; ketone such as acetone, methyl isobutyl ketone (MIBK), methyl ethyl ketone (MEK); polar aprotic such as N,N-dimethylformamide (DMF), dimethyl sulfoxide (DMSO), dimethyl acetamide (DMAc), N-methylpyrrolidone (NMP); polar protic such as alcoholic solvent C₁₋₄ alcohol such as methanol, ethanol, isopropanol, propanol, butanol and the like. The dissolution may be done at ambient temperature or at elevated temperature by heating the mixture at 40° C. to reflux. 2-butanol is taken in 5 to 20 volume by weight of the compound. The clear solution is filtered through hyflo bed to remove any fine particles undissolved. The filtrate is heated at 40° C. to reflux, preferably at 50-60° C. To this clear hot solution, hydrobromic acid is added. Hydrobromic acid may be provided as a gas or as a solution in an organic or aq. solvent. For example aq. hydrobromic acid, hydrobromic acid in acetic acid or hydrobromic acid gas in organic solvent can be used. It is taken in stoichiometric amount ranging from 1 to 2 mol equivalent of the compound. The reaction mixture is heated from 40° C. to reflux, preferably at 50-60° C. for 1 to 3 hour. The reaction mixture is gradually cooled to ambient temperature 25-35° C. and stirred for 1 to 3 hour. The precipitates are obtained. This process also encompass use of seed of form R3 to induce crystallization. The resulting precipitates were isolated from the suspension by the methods known in the art such as filtration or centrifugation. The solid is dried at 40-50° C. under vacuum. The solid was analysed by XRPD, DSC and TGA. The analytical data revealed that it is 2-butanol solvate of vortioxetine hydrobromide. It is designated as form R3. The vortioxetine free base used in above process is having HPLC purity 99.0 or above.

In another aspect, the present invention relates to a process for preparation of vortioxetine hydrobromide solvate form R3 comprising the steps of:

(a) providing a suspension of vortioxetine hydrobromide in 2-butanol or mixture of solvent containing 2-butanol

(b) isolating the resulting solid

In this process vortioxetine hydrobromide is added to 2-butanol or mixture of solvent containing 2-butanol to provide suspension. The mixture of solvent may be prepared by taking one or more cosolvent with 2-butanol. The cosolvent is as defined above. The suspension is heated at 40° C. to reflux, preferably at 50-60° C. for 1 to 3 hour. 2-butanol is taken in 5 to 20 volume by weight of the compound. The reaction mixture does not become clear solution. The reaction mixture is gradually cooled to ambient temperature 25-35° C. and stirred for 1 to 3 hour. The resulting precipitates were isolated from the suspension by the methods known in the art such as filtration or centrifugation. The solid is dried at 40-50° C. under vacuum. The solid was analysed by XRPD, DSC and TGA. The analytical data revealed that it is 2-butanol solvate Form R3 of vortioxetine hydrobromide. The vortioxetine hydrobromide used in above process is having HPLC purity 99.0 or above.

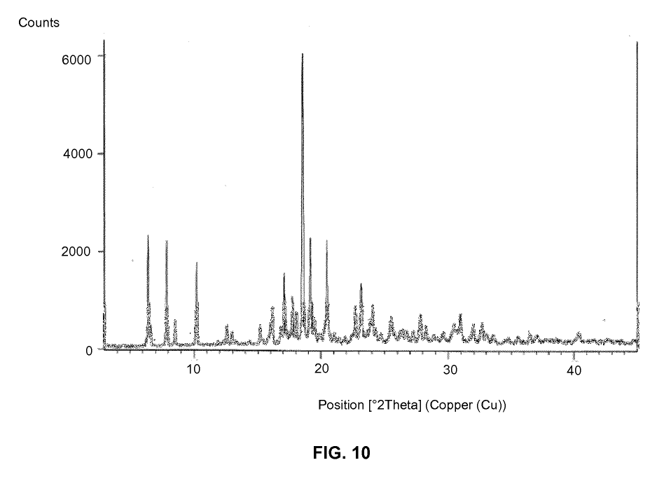

Vortioxetine hydrobromide form R4 can be characterized by XRPD patterns having peaks at about 7.8, 10.1, 18.5 and 20.4±0.2°2θ. Vortioxetine hydrobromide form R4 can be further characterized by XRPD reflections of peaks at 6.4, 7.8, 8.5, 10.1, 17.1, 17.7, 18.0, 18.5, 19.1, 20.4 and 22.7±0.2°2θ. A typical XRPD of vortioxetine hydrobromide form R4 as 2-butanol solvate is shown in FIG. 10.

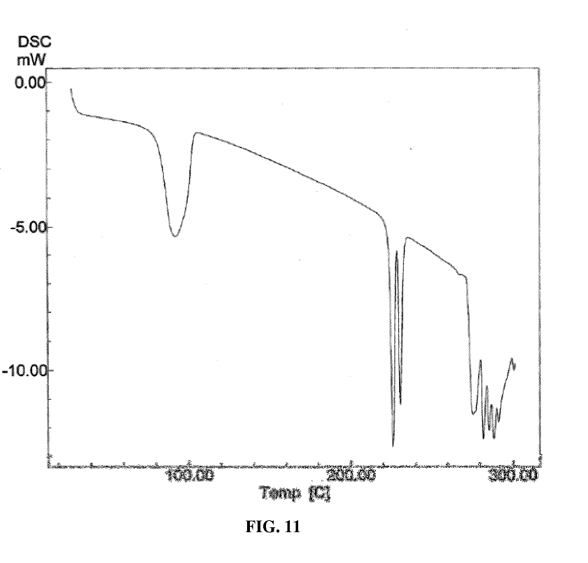

The DSC thermogram of vortioxetine hydrobromide form R4 is shown in FIG. 11. The DSC thermogram of vortioxetine hydrobromide form R4 is characterized by endothermic peaks at about 92.36° C., 226.18° C. and at about 230.77° C.

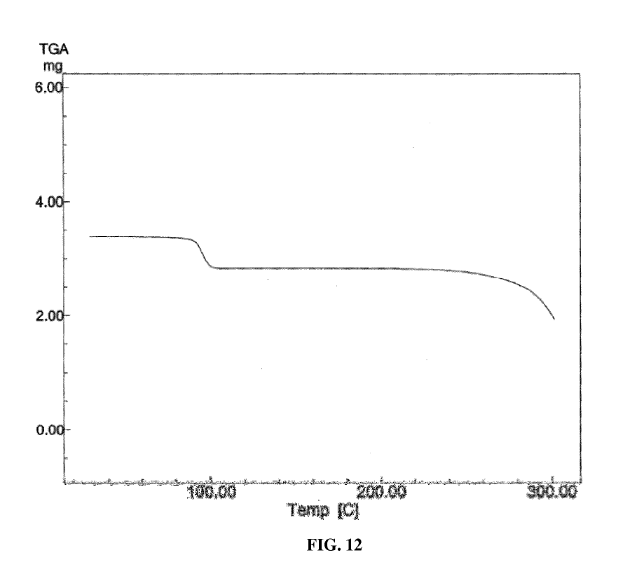

Vortioxetine hydrobromide form R4 shows weight loss of about 16.30% in TGA in the temperature range of between 80° C. and about 120° C. TGA for vortioxetine hydrobromide form R4 as 2-butanol solvate is shown in FIG. 12.

The present invention relates to a process for preparation of vortioxetine hydrobromide solvate form R4 comprising the steps of:

(a) providing a suspension of vortioxetine free base in 2-butanol or mixture of solvent containing 2-butanol

(b) adding hydrobromic acid to the above suspension and

(c) isolating the resulting solid

Vortioxetine free base is added in 2-butanol or mixture of solvent containing 2-butanol to provide suspension. The mixture of solvent may be prepared by taking one or more cosolvent with 2-butanol. The cosolvent is selected from organic solvent such as chlorinated hydrocarbons such as dichloromethane, dichloroethane, chloroform or carbon tetrachloride; aromatic hydrocarbon such as toluene, xylene; methyl tertbutyl ether (MTBE); nitrile such as acetonitrile; ester such as ethylacetate, isopropyl acetate; ketone such as acetone, methyl isobutyl ketone (MIBK), methyl ethyl ketone (MEK); polar aprotic such as N,N-dimethylformamide (DMF), dimethyl sulfoxide (DMSO), dimethyl acetamide (DMAc), N-methylpyrrolidone (NMP); polar protic such as alcoholic solvent C₁₋₄ alcohol such as methanol, ethanol, isopropanol, propanol, butanol and the like. The suspension is heated to at 50-60° C. to reflux for 20-30 min. The reaction mixture does not become clear solution. 2-butanol is taken in 5 to 20 volume by weight of the compound. To this hot suspension hydrobromic acid is added. Hydrobromic acid may be provided as a gas or as a solution in an organic or aq. solvent. For example aq. hydrobromic acid, hydrobromic acid in acetic acid or hydrobromic acid gas in organic solvent can be used. It is taken in stoichiometric amount ranging from 1 to 2 mol equivalent of the compound. The reaction mixture is heated from 40° C. to reflux, preferably at 50-60° C. for 1 to 3 hour. The reaction mixture is gradually cooled to ambient temperature 25-35° C. and stirred for 1 to 3 hour. The resulting precipitates were isolated from the suspension by the methods known in the art such as filtration or centrifugation. The solid is dried at 40-50° C. under vacuum. The solid was analysed by XRPD, DSC and TGA. The analytical data revealed that it is 2-butanol solvate of vortioxetine hydrobromide. It is designated as form R4. The vortioxetine free base used in above process is having HPLC purity 99.5 or above.

The following examples are given for the purpose of illustrating the present invention and should not be considered as limitation on the scope or spirit of the invention.

### EXAMPLE 1

Process for Preparation of Vortioxetine Hydrobromide t-Butanol Solvate Form R1

1-[2-(2,4-Dimethyl-phenylsulfanyl)-phenyl]-piperazine(100 g, 0.335 mol) was added in t-butanol (1000 ml) at 25-35° C. and heated to 50-60° C. to get clear solution. The solution was filtered to remove any fine particles and filtrate was heated up to 50-60° C. 47% aqueous hydrobromic acid (60.56 g, 0.352 mol) was added to the reaction mixture at 50-60° C. and stirred at the same temperature for 1 to 2 hour. Reaction mixture was gradually cooled and stirred at 25-35° C. for 1 to 2 hour. The precipitates were filtered and obtained solid was washed with tert-butanol (200 ml). The solid was dried at 45° C. under vacuum form 12 h to give the title product (142.0 g).

Yield: 93.0%

### EXAMPLE 2

Process for Preparation of Vortioxetine Hydrobromide t-Butanol Solvate Form R1

1-[2-(2,4-Dimethyl-phenylsulfanyl)-phenyl]-piperazine hydrobromide (100 g, 0.263 mol) was added in t-butanol (1000 ml) at 25-35° C. and heated to 45-55° C. for 1 to 2 hours to get suspension. The reaction mixture was gradually cooled and stirred at 25-35° C. for 1 to 2 hours. The precipitates were filtered and obtained solid was washed with tert-butanol (200 ml). The solid was dried at 45° C. under vacuum form 12 h to give the title product (107.0 g).

Yield: 90%

### EXAMPLE 3

Process for Preparation of Vortioxetine Hydrobromide t-Butanol Solvate Form R1 (From Tert Butanol:Methanol)

A mixture of 1-[2-(2,4-Dimethyl-phenylsulfanyl)-phenyl]-piperazine(100 g, 0.335 mol) in t-butanol (1500 ml) was heated at 60-70° C. Methanol (1000 ml) was added to the reaction mixture and stirred to get clear solution. The solution was filtered to remove any fine particles and filtrate was heated up to 60-70° C. 47% aqueous hydrobromic acid (60.56 g, 0.352 mol) was added to the reaction mixture at 60-70° C. and stirred at the same temperature for 1 to 2 hours. Reaction mixture was gradually cooled and stirred at 25-35° C. for 1 to 2 hours. The precipitates were filtered and obtained solid was washed with tert-butanol (200 ml). The solid was dried at 45° C. under vacuum form 12 h to give the title product (125.0 g).

Yield: 82%

### EXAMPLE 4

Process for Preparation of Vortioxetine Hydrobromide t-Butanol Solvate Form R1 (Tert Butanol:Dichloromethane)

A mixture of 1-[2-(2,4-Dimethyl-phenylsulfanyl)-phenyl]-piperazine(100 g, 0.335 mol) in dichloromethane (1500 ml) was heated at 35-45° C. Tert butanol (1000 ml) was added to the reaction mixture and stirred to get clear solution. The solution was filtered to remove any fine particles and filtrate was heated up to 40-45° C. 47% aqueous hydrobromic acid (60.56 g, 0.352 mol) was added to the reaction mixture at 40-45° C. and stirred at the same temperature for 1 to 2 hours. Reaction mixture was gradually cooled and stirred at 25-35° C. for 1 to 2 hours. The precipitates were filtered and obtained solid was washed with tert-butanol (200 ml). The solid was dried at 45° C. under vacuum form 12 h to give the title product (80.0 g).

Yield: 53%

### EXAMPLE 5

Process for Preparation of Vortioxetine Hydrobromide t-Butanol Solvate Form R2

1-[2-(2,4-Dimethyl-phenylsulfanyl)-phenyl]-piperazine(100 g, 0.335 mol) was added in t-butanol (1000 ml) at 25-35° C. and heated to 50-60° C. for 20-30 minutes to get suspension. 47% aqueous hydrobromic acid (60.56 g, 0.352 mol) was added to the reaction mixture at 50-60° C. and stirred at the same temperature for 1 to 2 hours. Reaction mixture was gradually cooled and stirred at 25-35° C. for 1 to 2 hours. The precipitates were filtered and obtained solid was washed with tert-butanol (200 ml). The solid was dried at 45° C. under vacuum form 12 h to give the title product (127.0 g).

Yield: 84.0%

### EXAMPLE 6

Process for Preparation of Vortioxetine Hydrobromide 2-Butanol Solvate Form R3

1-[2-(2,4-Dimethyl-phenylsulfanyl)-phenyl]-piperazine (100 g, 0.335 mol) was added in 2-butanol (1000 ml) at 25-35° C. and heated to 50-60° C. to get clear solution. The solution was filtered to remove any fine particles and filtrate was heated up to 50-60° C. 47% aqueous hydrobromic acid (60.56 g, 0.352 mol) was added to the reaction mixture at 50-60° C. and stirred at the same temperature for 1 to 2 h. Reaction mixture was gradually cooled and stirred at 25-35° C. for 1 to 2 h. The precipitates were filtered and obtained solid was washed with 2-butanol (200 ml). The solid was dried at 45° C. under vacuum for 12 h to give the title product (135.0 g).

Yield: 89.0%

### EXAMPLE 7

Process for Preparation of Vortioxetine Hydrobromide 2-Butanol Solvate Form R3

1-[2-(2,4-Dimethyl-phenylsulfanyl)-phenyl]-piperazine hydrobromide (100 g, 0.263 mol) was added in 2-butanol (1000 ml) at 25-35° C. and heated to 50-60° C. for 1 to 2 hours to get suspension. The reaction mixture was gradually cooled and stirred at 25-35° C. for 1 to 2 hours. The precipitates were filtered and obtained solid was washed with 2-butanol (200 ml). The solid was dried at 45° C. under vacuum for 12 hours to give the title product (107.0 g).

Yield: 90%

### EXAMPLE 8

Process for Preparation of Vortioxetine Hydrobromide 2-Butanol Solvate Form R4

1-[2-(2,4-Dimethyl-phenylsulfanyl)-phenyl]-piperazine(100 g, 0.335 mol) was added in 2-butanol (1000 ml) at 25-35° C. and heated to 50-60° C. for 20-30 minutes to get suspension. 47% aqueous hydrobromic acid (60.56 g, 0.352 mol) was added to the reaction mixture at 50-60° C. and stirred at the same temperature for 1 to 2 hours. Reaction mixture was gradually cooled and stirred at 25-35° C. for 1 to 2 hours. The precipitates were filtered and obtained solid was washed with 2-butanol (200 ml). The solid was dried at 45° C. under vacuum form 12 hours to give the title product (125.0 g).

Yield: 82.0%

1. A crystalline Form R1 of Vortioxetine hydrobromide tert-butanol solvate characterized by X-ray powder diffraction pattern having peaks at 6.7, 8.6, 15.9 and 19.1±0.2°2θ.

2. The crystalline Form R1 of Vortioxetine hydrobromide tert-butanol solvate according to claim 1, further characterized by X-ray powder diffraction pattern having peaks at 6.7, 7.9, 8.6, 13.5, 15.2, 15.9, 17.6, 19.1, 20.3, 20.8, 22.4 and 30.6±0.2°2θ.

3. The crystalline Form R1 of Vortioxetine hydrobromide tert-butanol solvate according to claim 1, having X-ray powder diffraction pattern as shown in FIG. 1.

4. The crystalline Form R1 of Vortioxetine hydrobromide tert-butanol solvate according to claim 1, characterized by DSC thermogram having endothermic peaks at 129.22° C. and at 226.9° C.

5. The crystalline Form R1 of Vortioxetine hydrobromide tert-butanol solvate according to claim 4, characterized by DSC thermogram as shown in FIG. 2.

6. The crystalline Form R1 of Vortioxetine hydrobromide tert-butanol solvate according to claim 1, characterized by loss on drying (LOD) of 16.48% in TGA in the temperature range of between 95° C. and 150° C.

7. The crystalline Form R1 of Vortioxetine hydrobromide tert-butanol solvate according to claim 6, characterized by TGA as shown in FIG. 3.

8. A process for preparation of crystalline Form R1 of Vortioxetine hydrobromide tert-butanol solvate according to claim 1, comprising the steps of:
(a) providing a solution of Vortioxetine free base in tert-butanol or mixture of solvent containing tert-butanol
(b) adding hydrobromic acid to the above solution and
(c) isolating the resulting solid.

9. The process for preparation of crystalline Form R1 of Vortioxetine hydrobromide tert-butanol solvate according to claim 1, comprising the steps of:
(a) providing a suspension of Vortioxetine hydrobromide in tert butanol or mixture of solvent containing tert-butanol
(b) isolating the resulting solid.

10. The process for preparation of crystalline Form R1 of Vortioxetine hydrobromide tert-butanol solvate according to claim 9, or wherein mixture of solvent is prepared by taking one or more co-solvent with tert-butanol wherein co-solvent is selected from chlorinated hydrocarbons; aromatic hydrocarbon; nitrile; ester; ketone; polar aprotic; polar protic; C₁₋₄ alcohol solvent.

11. A crystalline Form R2 of Vortioxetine hydrobromide tert-butanol solvate characterized by X-ray powder diffraction pattern having peaks at 7.5, 10.0, 18.1 and 20.2±0.2°2θ.

12. The crystalline Form R2 of Vortioxetine hydrobromide tert-butanol solvate according to claim 11, further characterized by X-ray powder diffraction pattern having peaks at 6.6, 7.5, 10.0, 13.3, 16.5, 18.1, 19.0, 19.1, 20.2, 22.5, 28.0 and 30.6±0.2°2θ.

13. The crystalline Form R2 of Vortioxetine hydrobromide tert-butanol solvate according to claim 11, having X-ray powder diffraction pattern as shown in FIG. 4.

14. The crystalline Form R2 of Vortioxetine hydrobromide tert-butanol solvate according to claim 11, characterized by DSC thermogram having endothermic peaks at 132.80° C. and at 226.51° C.

15. The crystalline Form R2 of Vortioxetine hydrobromide tert-butanol solvate according to claim 14, characterized by DSC thermogram as shown in FIG. 5.

16. The crystalline Form R2 of Vortioxetine hydrobromide tert-butanol solvate according to claim 11, characterized by loss on drying (LOD) of 16.45% in TGA in the temperature range of between 95° C. and 150° C.

17. The crystalline Form R2 of Vortioxetine hydrobromide tert-butanol solvate according to claim 16, characterized by TGA as shown in FIG. 6.

18. A process for preparation of crystalline Form R2 of Vortioxetine hydrobromide tert-butanol solvate according to claim 11, comprising the steps of:
(a) providing a suspension of Vortioxetine free base in tert-butanol or mixture of solvent containing tert-butanol
(b) adding hydrobromic acid to the above suspension and
(c) isolating the resulting solid.

19. The process for preparation of crystalline Form R2 of Vortioxetine hydrobromide tert-butanol solvate according to claim 18, wherein mixture of solvent is prepared by taking one or more co-solvent with tert-butanol wherein co-solvent is selected from chlorinated hydrocarbons; aromatic hydrocarbon; nitrile; ester; ketone; polar aprotic; polar protic; C₁₋₄ alcohol solvent.

20. The process for preparation of crystalline Form R1 of Vortioxetine hydrobromide tert-butanol solvate according to claim 8, wherein mixture of solvent is prepared by taking one or more co-solvent with tert-butanol wherein co-solvent is selected from chlorinated hydrocarbons; aromatic hydrocarbon; nitrile; ester; ketone; polar aprotic; polar protic; C₁₋₄ alcohol solvent.
